(19) United States
(12) Patent Application Publication
(10) Pub. No.: US 2007/0038117 A1
(43) Pub. Date: Feb. 15, 2007

(54) Multi-spectral imaging endoscope system

(76) Inventor: John L. Bala, Pomfret Center, CT (US)

Correspondence Address: ALIX YALE & RISTAS LLP, 750 MAIN STREET, SUITE 1400, HARTFORD, CT, 06103, US

(21) Appl. No.: 11/189,661

(22) Filed: Jul. 26, 2005

(51) Int. Cl. A61B 6/00

(52) U.S. Cl. 600/476

(57) ABSTRACT

Light is introduced into the body through an endoscope illumination system, which is capable of passing both UV and visible radiation through an illumination pathway. An image can then be viewed in real time, by eye or with an electronic imaging camera and displayed on a video monitor used by the surgeon. Dyes which are activated by the UV radiation generate images that can be viewed by the endoscope in the visible spectrum and recorded by eye, electronic camera or other recording devices that can process visual images.

### FIELD OF THE INVENTION

The present invention generally relates to surgical devices. In particular, the invention relates to an endoscope having therapeutic interventional capability combined with imaging elements.

### BACKGROUND OF THE INVENTION

The ability to view interior portions of a patient's body during a surgical medical procedure is invaluable for efficacious surgical intervention. Conventionally, devices for viewing the interior of a patient's body during a surgical procedure utilize light guides. These conventional light guides allow areas within a patient's body cavities to be both illuminated and visualized through an eyepiece. These conventional systems utilize continuous (CW) light sources that are coupled to an illumination conduit by a light guide and an optical connector located at or near the top of the illumination device. The designers of light sources for use in conventional systems are typically concerned with only light in the visible wavelengths.

Imaging dyes are conventionally utilized by injection of the dyes into the blood and/or lymphatic system and in some cases into specific tissues such that the dyes can be imaged by, for example, X-ray or MRI apparatus. The resulting X-ray or MRI images are subsequently captured, for example, by photography or other storage means. However, there is no conventional means for real-time capture and processing of internal images during an endoscopic surgical procedure. In addition, conventional imaging techniques such as X-ray and MRI are not suitable for use in conjunction with endoscopes.

Typically, imaging dyes are best utilized with ultra violet light sources. However, typical endoscopes do not transmit deep into the ultra violet region of the light spectrum because of, among other things, the use of fused silica as the transmitting medium.

Current endoscopes cannot readily combine visual imaging and therapeutic intervention because their light source must be continuous; their fiber optic bandwith is limited; and their optics are inefficient, responding only to light between 400-700 nm. The multi-spectral endoscope uses pulsed xenon flashtubes which offer a broad optical spectrum (190-1200 nm) and which generate high-powered micro-second light pulses that convert non-visible light into visual images. These images can become visible with the use of photodynamic diagnostic dyes, IR sensors, or image converters. Multiplexing technology can also direct laser energy for ablation/coagulation by sharing the fiber optic illumination pathway into the body between imaging technology and therapeutic intervention capability. Pulsed xenon's UV output can directly kill some infectious bacteria in seconds; it can also identify thermal variations in solid tissue temperature. The IR and UV spectrum may be able to delineate solid tissue from blood vessels, as well as allow visualization within blood vessels or through smoke or fluid.

The multi-spectral endoscope uses optical concepts that replace up to 22 optical elements with a single component to increase the transfer efficiency and resolution of visual, UV and IR images. It can be equipped with different, interchangeable, low-cost, reusable or disposable illuminators which can be optimized for a given surgical procedure.

### SUMMARY OF THE INVENTION

Briefly stated, the present invention in a preferred form is generally directed toward an endoscopic device utilizing pulse xenon technology to produce wavelengths of light within the UV spectrum in order to provide real-time and/or stored differential imaging of internal tissues, fluid pathways and areas having a UV dye present. The endoscopic device includes a probe having a distal end and a proximal end. A shaft which includes an optical transmissive material is located between the distal and proximal ends. The optical transmissive material provides an optical pathway along the length of the shaft. The optical pathway can selectively be placed in transmissive communication with an image processing and/or capture system.

Associated with the probe is an illuminator having an illumination pathway capable of being in selective transmissive communication with a light source. The illuminator in some cases may include a barrier element capable of isolating portions of the endoscopic device. The illuminator may also be changeable and/or disposable and may include transmissive fiber optical material to bring specific wavelengths of light into the body of a patient.

An object of the present invention is to provide a new and improved imaging system which employs pulsed xenon illumination and the imaging of tissue, fluid pathways and/or areas containing UV dye within a patient's body.

An object of the present invention is to provide an endoscope having a reusable, removable, and/or disposable illuminator for transmission of light energy.

Another object of the present invention is to provide a reusable coherent fiber optic imaging bundle in which an image is transmitted from a proximal end of the device to a distal end of the device, wherein the coherent fiber optic bundle may be covered with a flexible cladding.

Another object of the present invention is to provide an optical system to selectively illuminate imaging dye, such that the dye may fluoresce or otherwise become detectable by an endoscope and visually displayed.

A further object of the present invention is to provide barrier elements that operatively isolate portions of the endoscope from contact with the patient and/or the user.

### BRIEF DESCRIPTION OF THE DRAWINGS

The present invention may be better understood and its numerous objects and advantages will become apparent to those skilled in the art by reference to the accompanying drawings in which:

FIG. 1 is a simplified partial perspective side view of a multi-spectral imaging flexible endoscope probe in accordance with the present invention.

FIG. 2 is a simplified perspective view of a disposable sheath with barrier element for use with a multi-spectral imaging endoscope system in accordance with the present invention.

FIG. 3 is a simplified partial perspective side view of a multi-spectral imaging endoscope having a rigid probe in accordance with the present invention.

FIG. 4 is a simplified partial perspective side view of a multi-spectral imaging endoscope which includes a disposable sheath and deployed barrier element in accordance with the present invention.

FIG. 5 is a simplified exploded view of a multi-spectral imaging endoscope with associated imaging system elements in accordance with the present invention.

FIG. 6 is a simplified end sectional view of an imaging element for use with components of a multi-spectral imaging endoscope as shown in FIG. 5.

FIG. 7 is a simplified side view of disposable plastic illuminator having an non-deployed barrier element for use with a multi-spectral imaging endoscope in accordance with the present invention.

FIG. 8 is a simplified sectional end view of disposable plastic illuminator for use with components of a multi-spectral imaging endoscope as shown in FIG. 7.

FIG. 9 is a graph of relative irradiance versus wavelength expressed in nanometers of a xenon flashtube.

FIG. 10 is a simplified perspective view of an endoscope imaging system in accordance with the present invention.

FIGS. 11A and 11B show a side and a top view of an endoscope having a rigid rod configuration in accordance with the present invention.

### DETAILED DESCRIPTION OF THE INVENTION

With reference to the drawings wherein like numerals represent like parts throughout the several figures, a multi-spectral endoscope in accordance with the invention is designated by the numeral 10. The multi-spectral endoscope 10, as shown in FIG. 4, can be used for medical bioimaging within, for example, a patient's abdominal cavity to enhance the visualization of areas of interest. For example, a site of interest located within a patient's abdominal cavity is illuminated with UV light. The site of interest can also be associated with structure, tissue, or fluid which when illuminated with UV light can be distinguished from the surrounding field of view. In addition, UV dye can be used in conjunction with the UV light illumination. The UV dye can, for example, be locally or systematically injected into the patient in order to image structures of interest. The multi-spectral feature refers to illumination in at least the UV and the visual (VIS) ranges.

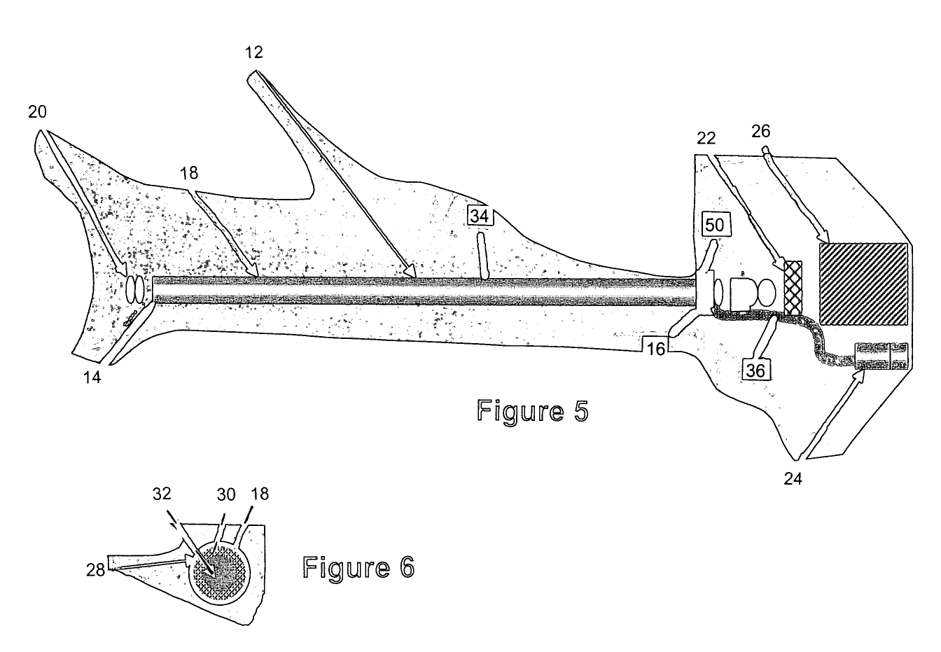

In one embodiment of the present invention, as shown in FIG. 5, the multi-spectral endoscope 10 includes a probe 12. The probe 12 has a distal end 14 and a proximal end 16. A shaft 18 is located between the distal end 14 and the proximate end 16. The shaft 18, as shown in FIG. 6, may have an outer encasement composed of, for example, living metal, plastic, and/or other material well known in the art of optical transmissive material encasement materials. In addition, the sheath may operate to optically isolate portions of the probe 12 from the illuminator 38. The shaft 18 includes an optical transmissive material 30 which defines at least a portion of an optical pathway 32. The optical pathway 32 extends substantially between the distal end 14 and proximal end 16 of the shaft 18. For example, the transmissive material 30 may be bundled fiber optical elements that extend substantially between the distal end 14 and proximal end 16 of the shaft 18.

In one embodiment of the present invention, the distal end 14 of the probe 12 is associated with at least one optical lens 20. The optical lens 20, for example, collimates the light relative to the optical pathway 32. The lens 20 may thus operate to advantageously gather and direct light into the optical pathway 32. The light may be in the visible or non-visible spectrum. For example, light which is produced or reflected from a structure or dye may enter the pathway 32.

It should be noted that a fiber optic annulus 28 associated with the pathway 32 provides transmissive conductivity to a remote location such as a camera 26. The fiber optic annulus, as shown in FIGS. 5 and 6 may substantially surround a portion of the proximal end 16 of the probe 12.

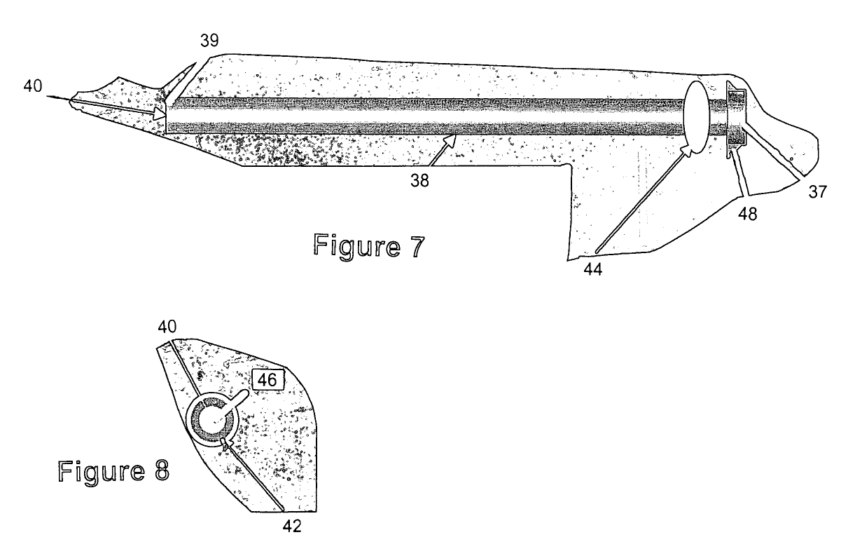

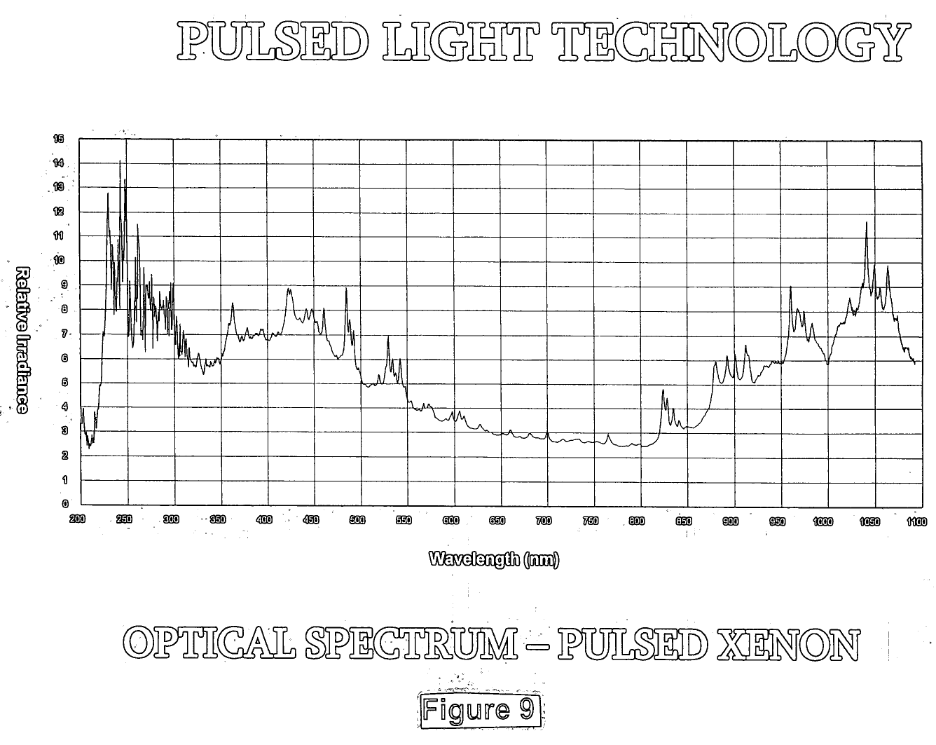

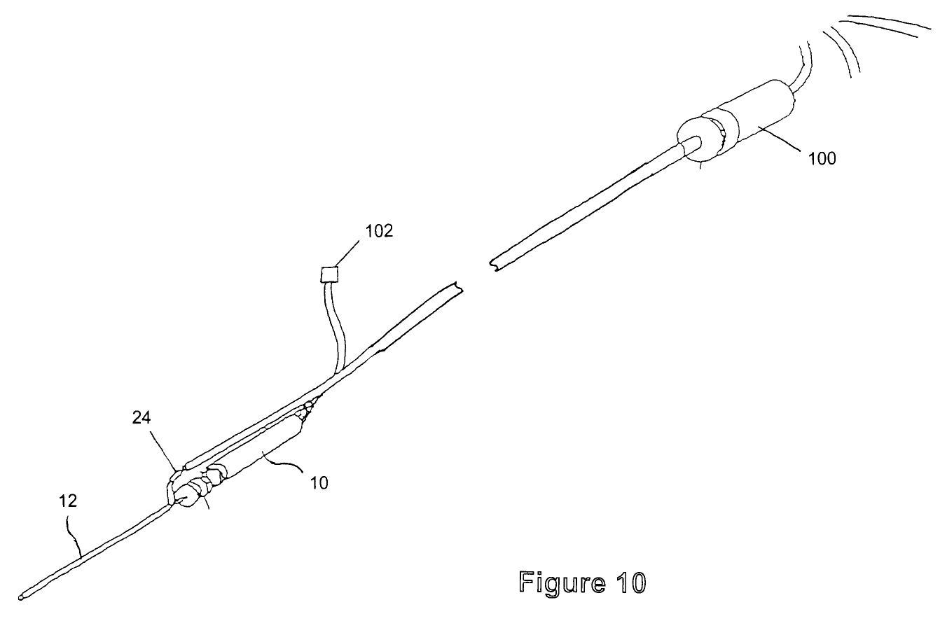

In one embodiment of the present invention, as shown in FIG. 7, the multi-spectral endoscope 10 includes an illuminator 38 having a distal end 39 and a proximal end 37. The illuminator 38 is preferably configured to receive a portion of the probe 12. For example, the illuminator 38 has, as shown in FIG. 8, a passage 46 for receiving a portion of the probe 12. The illuminator 38 also includes an illumination pathway 42. For example, fiber optical elements, such as quartz fiber optics, may form the illumination pathway 42. In addition, in some cases the user utilizes dyes that operate at or near the visible spectrum in a high energy form of the flashtube 100, as shown in FIG. 10. The flashtube 100 will provide an abundance of UV energy (FIG. 9) to expose/fluoresce the dyes which may allow the use of fused silica instead of other materials such as quartz. However, it should be noted that UV transmissive plastic fiber based optical materials such as Zeanor™, may also be utilized to form the illumination pathway 42.

In one embodiment of the present invention, as shown in FIG. 5, the illuminator 38, at the proximal end 37, interfaces with an annulus formed by, for example, a circular array of interface fiber optics. The annulus 48 provides a transmissive bridge between the flashtube 100 (FIG. 10) and the illuminator 38 such that light can be directed through the illumination pathway 42 and out into the body.

In one embodiment of the present invention, as shown in FIGS. 4 and 7, a barrier element 44 is positioned proximate the optical engagement end 37 of the illuminator 38. For example, the barrier element 44 can be configured as a rolled flexible tubular material which can be unrolled over portions of the UV endoscope 10 to insure that, for example, the probe 12 and/or elements of the handle 110 remain in a sterile field. The barrier element 44 thereby, among other things, allows the probe 12 to be used in multiple procedures without the necessity of re-sterilization. The barrier element 44 may be formed from, for example, extruded latex, polyethelene or other flexible extrudable materials.

In one embodiment of the present invention, as shown in FIG. 8, the illuminator 38 isolates the probe. For example, the illuminator 38 may biologically, chemically, and/or electrically isolate the probe 12 from the exterior environment. A sealing lens 40 preferably covers the probe distal end 14 and the illuminator distal end 39 of the illuminator 38.

In one embodiment of the present invention, as shown in FIG. 5, the probe 12 interfaces with, for example, an electronic camera 26. The interface can be configured to be directly interfaced with the camera 26. Direct interface can, in some instances, improve the optical transmission through the unit. Direct interface also allows for advantageous camera imaging, but provides for an optional eyepiece 102 (FIG. 10). The eyepiece 102 could be used in emergency situations by allowing the surgeon to make direct visual observations through the eyepiece 102.

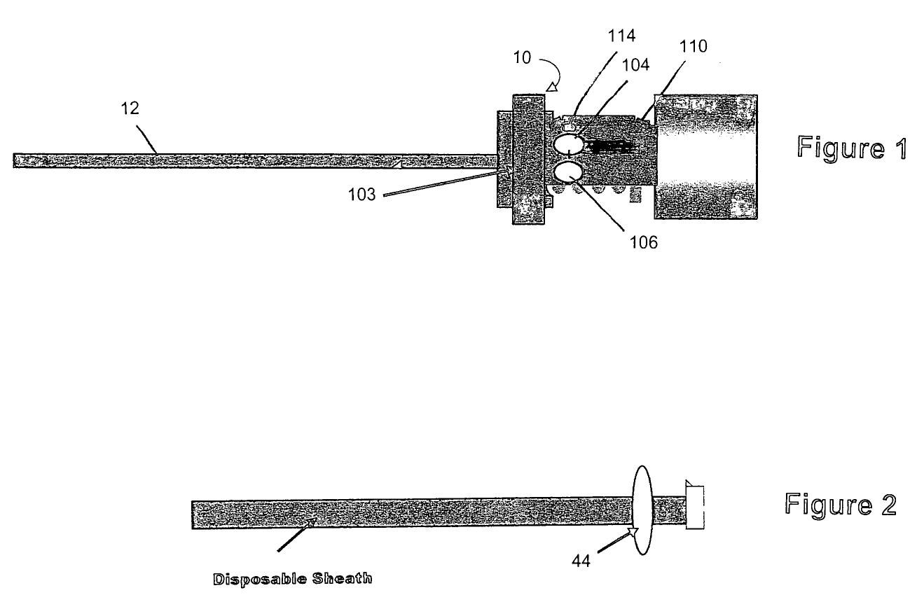

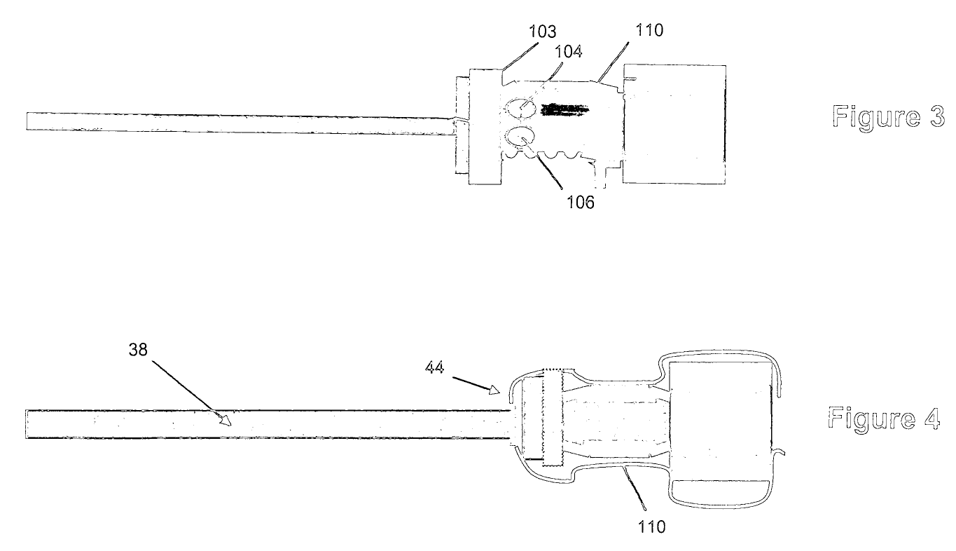

In one embodiment of the present invention, the multi-spectral endoscope system 10 includes controls that allow the surgeon to electronically increase the brightness of the image or to expand or contract the size of the image electronically. For example, as shown in FIGS. 1 and 3, a brightness increase control 104 and a brightness decrease control 106 is present on the handle 110. The brightness increase control 104 and brightness decrease control 106 advantageously allow the surgeon to increase or decrease light levels as the endoscope is relationally moved relative to a patient's body. This control can be advantageously, for example, accomplished without the assistance of an assistant. In addition, a focus control 103 is present between the probe and the handle 110. The focus control 103 is operatively associated with optical elements 50 (FIG. 5). The focus control 103 allows the operator to acquire the best focus for a given camera or image display. In addition, an image can be magnified by actuation of, for example, a zoom control 114 present on the handle 110. The zoom control is operatively connected with a digital counter 50 for electronic magnification and/or with the camera 26 for such things as digital magnification.

In one embodiment of the present invention, the endoscope handle 110 is ergonomically configured such that a user can easily and comfortably access the control features of the endoscope. The ergonomic configuration is such that the device can be held like a knife which, among other things, allows for more precise control and a reduction in the fatigue to the device operator.

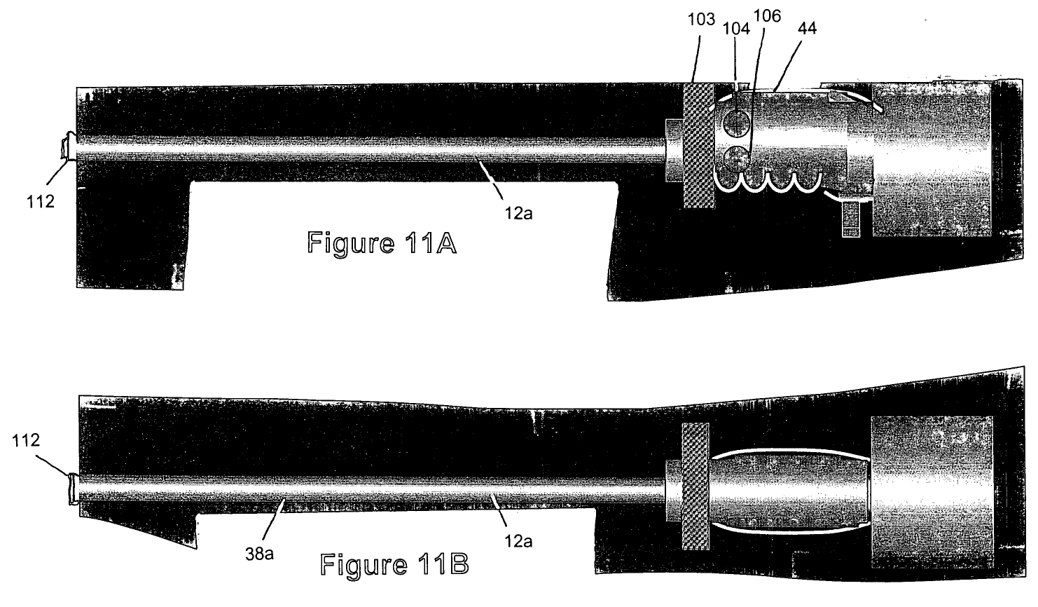

In one embodiment of the present invention, as shown in FIGS. 11A and 11B, the multi-spectral endoscope 10 may be configured with a rigid probe 12a. The rigid probe 12a includes an illuminator 38a can be configured to include angular viewing elements 112. The angular viewing elements 122 provide the ability to, for example, view at angles other than zero degrees. This capability thereby enables the viewing of points about 360 degrees without the need to move the camera and/or fiber optic cables. In addition, angular viewing elements 122 having different viewing characteristics may be interchangeable such that the user can interchange angles during a procedure without any set up or changes to the system.

The multi-spectral endoscope utilizes the full optical spectrum of illumination for visual and activated imagery, for laser ablation and coagulation, and for both diagnosis and therapy using rigid or flexible devices. This endoscope is designed to offer today's standard capabilities with incremental technical expansion as new procedures and features become FDA approved. This technology can be applied to flexible endoscopes, arthroscopes and other, more specialized scopes for otolaryncology, urology and cystoscopy, gynecology, spinal surgery and more.

While preferred embodiments of the foregoing invention have been set forth for purposes of illustration, the foregoing description should not be deemed a limitation of the invention herein. Accordingly, various modifications, adaptations and alternatives may occur to one skilled in the art without departing from the spirit and scope of the present invention.

What is claimed is:

1. A multi-spectral endoscope system comprising:
a light source which produces both UV and visible light;
a viewing element;
a handle;
a probe associated with the handle, said probe having a distal end and a proximal end, the probe having a light transmissive pathway extending from the distal end to the proximal end, said transmissive pathway in optical communication with the viewing element; and
an illuminator associated with the probe, said illuminator having a distal end and a proximal end, the illuminator having a UV and visible light pathway extending between the distal end and the proximate end and in optical communication with said UV light.

2. The multi-spectral endoscope system of claim 1, wherein the illuminator receives a portion of the probe.

3. The multi-spectral endoscope system of claim 2, wherein the UV-vis light pathway substantially surrounds a central opening configured to receive a portion of the probe.

4. The multi-spectral endoscope system of claim 3, wherein at least one of the light transmissive pathway and the light pathway is flexible.

5. The multi-spectral endoscope system of claim 3, wherein at least one of the light transmissive pathway and the light pathway is rigid.

6. The multi-spectral endoscope system of claim 1, wherein the illuminator biologically isolates the probe.

7. The multi-spectral endoscope system of claim 1, wherein the illuminator includes a barrier element that extends over an outer surface of the illuminator.

8. The multi-spectral endoscope system of claim 1, wherein the illuminator includes a barrier material for selective isolation of a portion of the handle.

9. The multi-spectral endoscope system of claim 1, further comprising a sealing lens at the distal end of said illuminator, said sealing lens configured to isolate a portion of the probe and to allow the transmission of UV and visible light from the illuminator and to allow light transmission into the probe light transmissive pathway.

10. The multi-spectral endoscope system of claim 1, wherein the probe includes a fiber optic annulus at the proximal end, the fiber optic annulus being in optical communication with the viewing element.

11. The multi-spectral endoscope system of claim 1, wherein the viewing element includes a camera.

12. The multi-spectral endoscope system of claim 1, wherein the viewing element includes an eyepiece.

13. The multi-spectral endoscope system of claim 1, further including a focus control, the focus control being disposed between the distal end of the probe and the viewing element.

14. The multi-spectral endoscope system of claim 1, wherein said proximal end of the probe is selectively engagable with the handle.

15. An imaging system comprising:
a substantially cylindrical probe member having a light pathway defined between a light input end and a light output portion;
an illuminator having a UV-VIS light pathway defined between a UV-VIS light input portion proximate a proximal end and a UV-VIS light output proximate the distal end, said illuminator having a central opening configured to receive the probe;
a light source capable of producing light in the UV-VIS light spectrum, said light source communicatively associated with the light input portion of the illuminator; and
an imager communicatively associated with the light output portion of the probe, said imager including a viewing element.

16. The imaging system of claim 15, wherein the substantially cylindrical probe has an optical core and an illuminator disposed about a portion of the optical core, said illuminator configured to optically isolate the core from the UV-VIS pathway of the illuminator.

17. The imaging system of claim 15, wherein the light source is a pulsed xenon flashtube.

18. The imaging system of claim 15, wherein the light output portion of the probe is optically associated with an annulus.

19. The imaging system of claim 15, wherein the light input end of the probe is optically associated with at least one lens.

20. The imaging system of claim 15, wherein a focus control is disposed between the light output portion of the probe and the imager.

21. A method of imaging portions of a patient having a pre-applied UV activated dye at selective tissue thereof comprising:
generating light in the UV-VIS spectrum;
transmitting said light to a probe having a proximal end and a distal end;
introducing said probe distal end into the vicinity of said tissue;
illuminating said dye and tissue with said light to activate said dye; and
transmitting an image of said tissue through said probe.

22. The method of claim 21 wherein said UV-VIS light is generated by a pulsed xenon light source.

23. The method of claim 21 wherein said image is transmitted to a camera.

24. The method of claim 21 further comprising transmitting light through an annular array of interface fiber optics.
